(19) United States
(12) Patent Application Publication
(10) Pub. No.: US 2025/0000543 A1
(43) Pub. Date: Jan. 2, 2025

(54) SURGICAL BLADE FOR PERFORMING CUTTING AND SUCTION

(71) Applicant: KING FAISAL UNIVERSITY, Al-Ahsa (SA)

(72) Inventor: ABDULRAHMAN SALEH ALMULHIM, Al-Ahsa (SA)

(21) Appl. No.: 18/214,794

(22) Filed: Jun. 27, 2023

(51) Int. Cl. A61B 17/3211

(52) U.S. Cl. CPC A61B 17/3211; A61B 2217/005

(57) ABSTRACT

The surgical blade for performing cutting and suction includes a handle portion having opposed proximal and distal ends, with a port defined in the distal end thereof. The surgical blade also includes a blade portion secured to the proximal end of the handle portion and extending proximally therefrom. The blade portion has at least one opening formed through at least one side surface thereof. A channel is continuously formed through the handle portion and into the blade portion. A proximal end of the channel is closed and is located within the blade portion, and a distal end of the channel is open and is in open communication with the port. The at least one opening is in open communication with the channel, and the port is adapted for connection to a source of suction.

### BACKGROUND OF THE INVENTION

### Field of the Invention

The disclosure of the present patent application relates to surgical blades, such as scalpels and the like, and particularly to a surgical blade which is adapted for both cutting and performing suction of bodily fluids and the like.

### Description of Related Art

Cutting during medical procedures, such as the incising and draining of abscesses and cystic lesions, for example, typically result in the expulsion of bodily fluids, such as pus and blood. During such procedures, the medical practitioner typically pauses to wipe away the fluids with medical gauze or apply suction to remove the fluids. These interruptions to remove the fluids are so common that they are typically considered to be part of the procedure. It would be desirable to be able to remove the fluids without requiring the medical practitioner to pause the active parts of the procedure. Thus, a surgical blade for performing cutting and suction solving the aforementioned problems is desired.

### SUMMARY OF THE INVENTION

The surgical blade for performing cutting and suction is a surgical blade, such as a scalpel or the like, which is adapted for not only performing cutting but for also performing suction of bodily fluids, such as pus, and other unwanted or interfering fluids during surgical cutting procedures. The surgical blade for performing cutting and suction includes a handle portion having opposed proximal and distal ends, with a port defined in the distal end thereof. The surgical blade for performing cutting and suction also includes a blade portion secured to the proximal end of the handle portion and extending proximally therefrom. The blade portion has at least one opening formed through at least one side surface thereof. Additionally, a channel is continuously formed through the handle portion and into the blade portion. A proximal end of the channel is closed and is located within the blade portion, and a distal end of the channel is open and is in open communication with the port. The at least one opening is in open communication with the channel, and the port is adapted for connection to a source of suction, such as an external vacuum pump or the like.

These and other features of the present subject matter will become readily apparent upon further review of the following specification.

### BRIEF DESCRIPTION OF DRAWINGS

FIG. 1 is a side view of a surgical blade for performing cutting and suction.

FIG. 2 is a side view in section of the surgical blade for performing cutting and suction.

Similar reference characters denote corresponding features consistently throughout the attached drawings.

### DETAILED DESCRIPTION

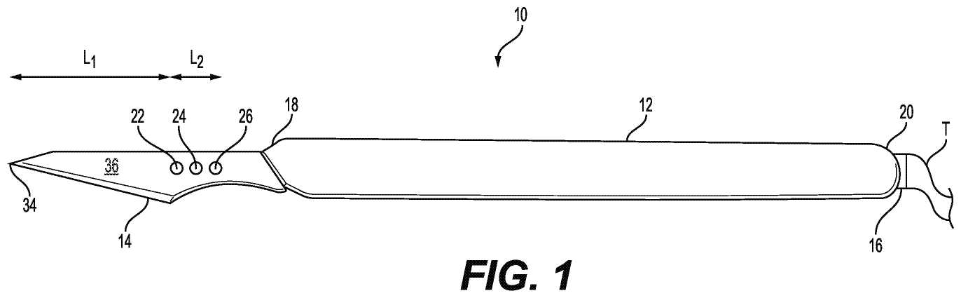

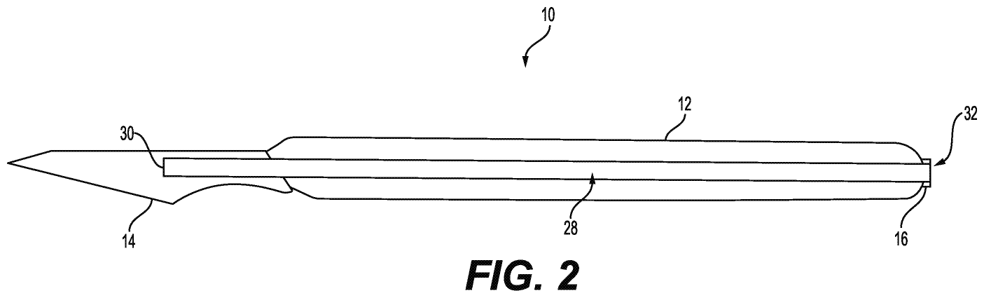

The surgical blade for performing cutting and suction 10 is a surgical blade, such as a scalpel or the like, which is adapted for not only performing cutting but for also performing suction of bodily fluids, such as pus, and other unwanted or interfering fluids during surgical cutting procedures. As shown in FIG. 1, the surgical blade for performing cutting and suction 10 includes a handle portion 12 having opposed proximal and distal ends 18, 20, respectively, with a port 16 defined in the distal end 20. A blade portion 14 is secured to the proximal end 18 of the handle portion 12 and extends proximally therefrom. As is well known in the field of scalpels and similar surgical cutting instruments, blade portion 14 may be integrally formed with handle portion 12 or may be attached thereto, either permanently or removably. Further, it should be understood that the overall configuration, shape and relative dimensions of the surgical blade for performing cutting and suction 10 are shown in FIGS. 1 and 2 for exemplary purposes only, and other configurations, shapes and relative dimensions fall within the scope of the contemplated subject matter. Similarly, it should be understood that blade portion 14 and handle portion 12 may be made from any suitable materials, such as surgical stainless steel or the like.

The blade portion 14 has at least one opening formed through at least one side surface thereof. In the non-limiting example of FIG. 1, three openings 22, 24, 26 are shown formed through a single side surface 36 of blade portion 14. However, it should be understood that any suitable number of openings may be included, and that one or more openings may also be formed through the other side of the blade portion 14. Further, it should be understood that the circular shape and relative dimensions of openings 22, 24, 26 are shown in FIG. 1 for exemplary purposes only and may be varied. Similarly, it should be understood that the spacing and relative locations of openings 22, 24, 26 are shown in FIG. 1 for exemplary purposes only.

In a particular non-limiting example, the proximal opening 22 may be located approximately 25 mm from the proximal tip 34 of blade portion 14, measured along the axis of the surgical blade 10 (indicated as length L1 in FIG. 1). As a further non-limiting example, the three openings 22, 24, 26 may be evenly spaced apart across a distance of approximately 10 mm, also measured along the axis of the surgical blade 10 (indicated as length L2 in FIG. 1). It should be understood that the surgical blade 10 may be any suitable type of surgical cutting tool or instrument, and that the scalpel configuration of FIGS. 1 and 2 is shown for exemplary purposes only. In the non-limiting example of FIGS. 1 and 2, corresponding to the non-limiting dimensions given above, the handle portion may have a length of approximately 8 cm. However, these dimensions are for illustrative purposes only and are not meant to be limiting to the scope of the present subject matter.

As shown in FIG. 2, a channel 28 is continuously formed through the handle portion 12 and at least part way into the blade portion 14. A proximal end 30 of the channel 28 is closed and is located within the blade portion 14, and a distal end 32 of the channel 28 is open and is in open communication with the port 16. The at least one opening 22, 24, 26 is in open communication with the channel 28, and the port 16 is adapted for connection to a source of suction, such as an external vacuum pump or the like (not shown), through tubing T or the like. It should be understood that port 16 may include any suitable type of connector or adapter for connection to the source of suction. In use, as the surgeon uses blade portion 14 to cut during a medical or surgical procedure, the source of suction may be turned on to suction bodily fluids, such as pus or blood, from the site of the incision, or to suction any other unwanted fluids, such as saline or the like, through the openings 22, 24, 26.

It is to be understood that the surgical blade for performing cutting and suction is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A surgical blade for performing cutting and suction, comprising:
a handle portion having opposed proximal and distal ends, a port being defined in the distal end of the handle portion; and
a blade portion secured to the proximal end of the handle portion and extending proximally therefrom, wherein the blade portion has at least one opening for fluid removal formed through at least one side surface thereof, the at least one opening for fluid removal formed a first distance from a proximal tip of the blade portion and close to where the blade portion is secured to the proximal end of the handle portion,
wherein a channel is continuously formed through the handle portion and into the blade portion, a proximal end of the channel being closed at the first distance from the proximal tip of the blade portion and being located within the blade portion, a distal end of the channel being open and being in open communication with the port,
wherein the at least one opening for fluid removal is in open communication with the channel,
wherein the port is adapted for connection to a source of suction,
wherein the at least one opening for fluid removal comprises a plurality of openings for fluid removal, and
wherein the plurality of openings for fluid removal are evenly spaced across a second distance.

2-3. (canceled)

4. The surgical blade for performing cutting and suction as recited in claim 1, wherein the second distance is 10 mm.

5. The surgical blade for performing cutting and suction as recited in claim 1, wherein the first distance is 25 mm.
